(19) United States
(12) Patent Application Publication
(10) Pub. No.: US 2019/0053949 A1
(43) Pub. Date: Feb. 21, 2019

(54) Eye Mask and Method of Using Same

(71) Applicant: Sylvia Morris, Indianapolis, IN (US)

(72) Inventor: Sylvia Morris, Indianapolis, IN (US)

(21) Appl. No.: 16/104,019

(22) Filed: Aug. 16, 2018

Related U.S. Application Data
(60) Provisional application No. 62/605,509, filed on Aug. 16, 2017.

(51) Int. Cl. A61F 9/04; A41D 27/20

(52) U.S. Cl. CPC A61F 9/04; A41D 27/20

(57) ABSTRACT

An eye mask having pockets configured to hold materials over a user's eyes.

### CROSS-REFERENCE TO RELATED APPLICATIONS

This application claims the benefit of U.S. Provisional Application No. 62/605,509 titled “Eye Mask Method,” to Sylvia Morris, filed Aug. 16, 2017, the entire disclosure of which is expressly incorporated by reference herein.

### FIELD OF THE DISCLOSURE

The present disclosure relates generally to eye masks.

### BACKGROUND AND BRIEF SUMMARY

Eye masks are used by those attempting to sleep or sleeping to at least partially block light from reaching the user's eyes.

According to the present disclosure, an eye mask is provided having a mask portion and a pair of spaces configured to hold materials over a user's eyes. A method of using the same is provided.

### BRIEF DESCRIPTION OF THE DRAWINGS

The aforementioned aspects and many of the intended advantages of this disclosure will grow to be appreciated at a greater level once references to the following accompanying illustrations are expounded upon.

FIG. 1 is a rear view of an eye mask having a pair of pockets defining a pair of spaces configured to hold material over a user's eyes;

FIG. 2 is a front view of the eye mask of FIG. 1; and

FIG. 3 is perspective view of the eye mask of FIG. 1 as worn by a user.

Corresponding reference characters indicate corresponding parts throughout the several views. The drawings represent embodiments of various features and components according to the present disclosure and, unless otherwise indicated, show the components in proportion to one another. The exemplifications set out herein are not to be construed as limiting the scope of the disclosure in any manner.

### DETAILED DESCRIPTION

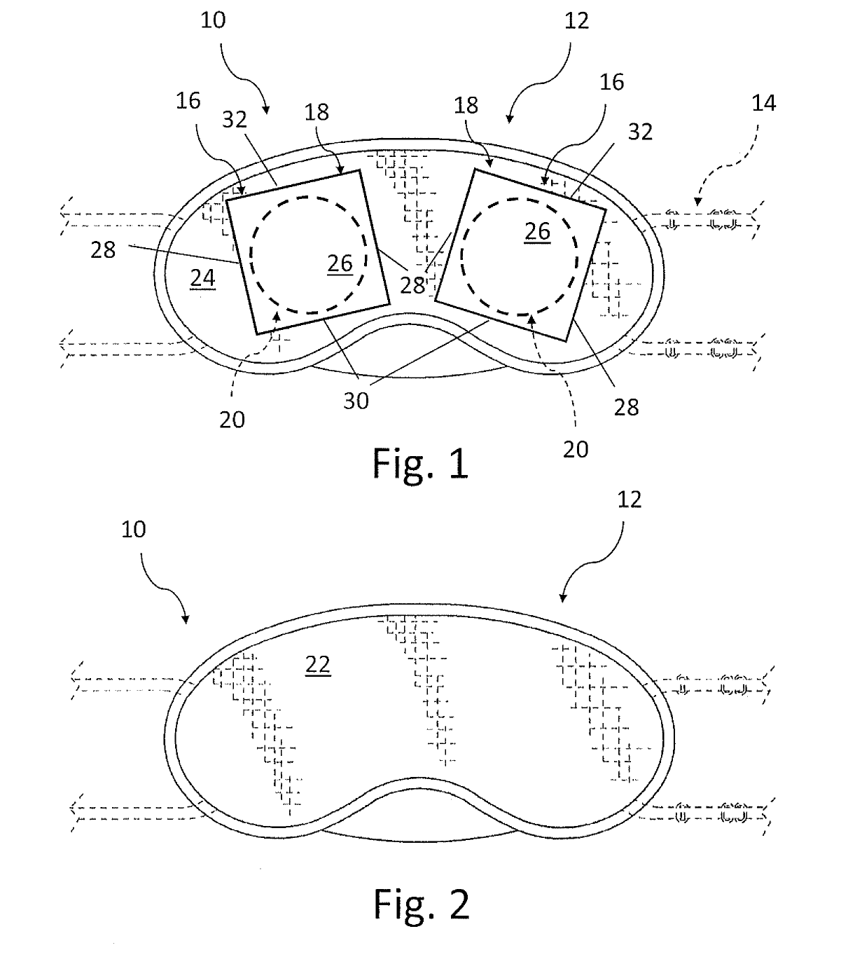

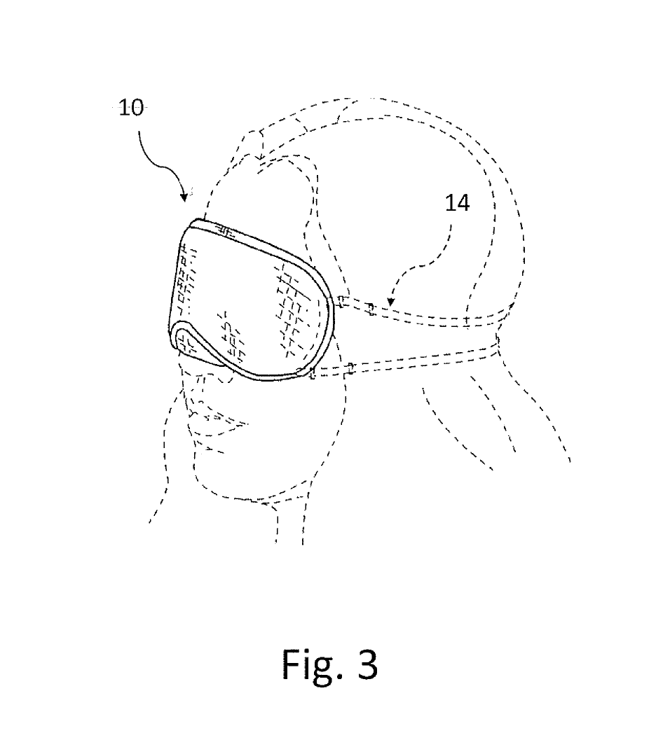

An eye mask 10 is shown in FIG. 1 having a mask portion 12, a pair of straps 14, and a pair of pockets 16 defining spaces 18 configured to hold materials 20 (shown in phantom). Mask portion 12 is made of an opaque fabric or other material to block light from passing there through. According to the preferred embodiment, mask portion 12 is 100% opaque (100% visible light blocking), but mask portion 12 may have other opacities, such as 99.9, 99.5, 99%, 98%, 97%, 95%, 92%, 90%, etc. Mask portion may including an outer fabric layer 22, an inner fabric layer 24, and padding (not shown) positioned between inner and outer fabric layers 22. Fabric layers 22, 24 may be made of satin, silk, cotton, or other materials and is preferable machine washable. Mask portion 12 is bean-shaped or hourglass-shaped and may have a nose portion.

Pockets 16 include an outer material 26 preferable made of a substantially liquid permeable fabric or material to allow fluids, such as water, to pass through. Pockets 16 further include an inner material (not shown but similarly sized to outer material 26) preferable made of a substantially liquid impermeable fabric or material to block the flow of fluids to mask portion 12. Pockets 16 may be stitched or otherwise coupled to mask portion 12 along sides 28 and bottom 30 leaving top 32 open. Each pocket 16 defines one of spaces 18 alone or in cooperation with mask portion 12 and are positioned to lay over a user's eyes when eye mask 10 is worn by a user. According to one embodiment, a single pocket is provided that defines the pair of spaces positioned to lay over a user's eyes.

According to a method of using eye mask 10, a user selects a material to be positioned over the eyes of the user during a therapy session. Such materials are typically organic, such as black, green, or herbal teas, rose petals, cucumber slices (shown in phantom in pockets 16), potatoes, apple cider or other vinegars, or other vegetation-based materials. However, other materials may also be selected, such as vitamins and minerals including vitamin A, C, E, K, iron, etc. Once the material(s) are selected, a user then places the materials into spaces 18 and positions mask 10 over their head so that pockets 16 are positioned over the user's eyes. Straps 14 are preferably elastic and used to secure eye mask 10 to the user's head. Liquids from the materials seep through outer material 26 onto the user's eye lids and other areas around the eyes while the inner material of pockets 16 blocks the fluid from flowing into mask portion 12. Eye mask 10 is worn by the user for a period of time, such as 1, 2, 3, 5, 7, 10, 15, 30, 30, 45, 60 minutes, etc., allowing materials to rest on the user's eye lids until mask 10 is removed. The materials can then be removed from pockets 18 and discarded. Mask 10 can then be cleaned for future use.

1. An eye mask including
a mask portion; and
a pair of spaces configured to hold material over a user's eyes.

2. The eye mask of claim 1, wherein the pocket spaces are defined by a pair of pockets coupled to the mask portion.

3. The eye mask of claim 1, further including a strap configured to hold the mask portion over the user's eyes.

4. A method for holding material over a user's eyes including the steps of
providing an eye mask having a pair of spaces,
inserting material into the pair of spaces,
placing the eye mask on a user with the material positioned over the users eyes.

5. The method of claim 4, where the material is tea.
